(19) United States
(12) Patent Application Publication
(10) Pub. No.: US 2024/0043366 A1
(43) Pub. Date: Feb. 8, 2024

(54) METHOD FOR CATALYTIC CONVERSION OF GLYCERIN INTO PRODUCTS OF HIGH ADDED VALUE, AND USE

(71) Applicants: PETRÓLEO BRASILEIRO S.A. – PETROBRAS, Rio de Janeiro (BR); UNIVERSIDADE FEDERAL DE MINAS GERAIS-UFMG, Belo Horizonte (BR)

(72) Inventors: Marcio DE FIGUEIREDO PORTILHO, Niterói (BR); Ana PACHELI HEITMANN RODRIGUES, Belo Horizonte (BR); Poliane CHAGAS, Belo Horizonte (BR); Cíntia DE CASTRO OLIVEIRA, Belo Horizonte (BR); Samuel MOURA BREDER, Belo Horizonte (BR); Alexander RANGEL BASTOS, Rio de Janeiro (BR); Luiz Carlos ALVES DE OLIVEIRA, Belo Horizonte (BR); Daniel LARA SANGIORGE, Belo Horizonte (BR)

(21) Appl. No.: 18/258,754

(22) PCT Filed: Nov. 25, 2021
(86) PCT No.: PCT/BR2021/050517

§ 371 Date: Jun. 21, 2023

(30) Foreign Application Priority Data
Dec. 22, 2020 (BR) 10 2020 026453 2

(51) Int. Cl. C07C 51/285; C07C 41/09; B01J 35/10; B01J 21/08; B01J 37/04; B01J 37/03

(52) U.S. Cl. CPC C07C 51/285; C07C 41/09; B01J 35/1028; B01J 21/08; B01J 37/04; B01J 37/031

(57) ABSTRACT

Disclosed is a catalyst based on synthetic silica, in a heterogeneous catalysis method, to promote the effective conversion of residual glycerin, resulting from the production of biodiesel, into formic acid with high selectivity and stability, in a continuous flow reaction. The conversion of residual glycerin occurs by homogeneous catalysis, by the action of components remaining from the synthesis of biodiesel, with the formation of major compounds, such as formic acid, cyclic ethers and diglycerol, in continuous flow and reflow reactions. The reaction can also be carried out by adding sodium salts in the homogeneous catalytic conversion process of commercial glycerin. The process values the residual glycerin, without the need for purification before its transformation into products with high added value, but of renewable origin, adding more interest and potential.

### FIELD OF THE INVENTION

The present invention addresses to obtaining compounds with high added value, from residual glycerin from the synthesis of biodiesel.

The method of this invention comprises conversions of glycerin by two catalytic processes: (i) heterogeneous catalysis, using a silica catalyst (without the use of metals), with acidic properties and presenting mesopores, and (ii) homogeneous catalysis, in which the used catalyst constitutes an impurity of the residual glycerin, upon the addition of an oxidizing agent. The products obtained may be used, for example, in applications of petrochemical interest, due to the characteristics of the formed molecules.

The method applied in this invention values the residual glycerin without the need for purification prior to its transformation by the employed reactions.

### DESCRIPTION OF THE STATE OF THE ART

Improving biodiesel production attracts widespread interest worldwide, not being different in the Brazilian case, since its production still has some limitations, such as, for example, the generation of large amounts of glycerin as a by-product.

In December 2004, the Brazilian federal government instituted the National Program for the Production and Use of Biodiesel (PNPB), which aimed at introducing biodiesel into the Brazilian energy matrix. As of 2004, its addition to fossil diesel was experimental, with 2% of biodiesel added to mineral diesel. However, in January 2008, the addition of 2% biodiesel became mandatory, giving rise to the fuel called B2 (ANP, 2018). This percentage was gradually increased, and in March 2018 the mandatory percentage became 10%. Several studies have already been carried out so that this percentage could reach 15% in 2019 (ANP, 2018).

In face of this context, there is a constant search for new technologies to significantly contribute to this important objective, since the compounds obtained from the conversion of residual glycerin, a by-product of biodiesel, have a high market value.

The present invention addresses to obtaining compounds with high added value, from residual glycerin from the synthesis of biodiesel. The method of this invention comprises conversions of glycerin by heterogeneous catalysis, using a silica catalyst without the presence of metals and conversion by homogeneous catalysis, wherein the catalyst used constitutes an impurity of the residual glycerin, through the addition of an oxidizing agent.

Several studies have been carried out, highlighting the importance of obtaining new products with greater added value from glycerol. In “Selective oxidation of glycerol to formic acid catalyzed by iron salts”, Catalysis Communications, volume 84, 24 May 2016, p. 1-4, the glycerol conversion reactions were carried out by means of a homogeneous catalysis, in which an iron-based catalyst solubilized in acetonitrile was employed in the conversion of commercial glycerol, using hydrogen peroxide as an oxidizing agent. In the present invention, it was not necessary to add a catalyst in the homogeneous catalysis, since the impurities present in the residual glycerol, from the production of biodiesel, in the presence of hydrogen peroxide, were capable of promoting the conversion of glycerol into high associated value products.

The paper “Oxidation of hydroxyacetone (acetol) with hydrogen peroxide in acetonitrile solution catalyzed by iron (III) chloride”, Journal of Molecular Catalysis A: Chemical, volume 422, 11 Feb. 2016, p. 103-114, addresses to the oxidation of acetol (1-hydroxypropanone) via homogeneous catalysis. The main objective was to produce compounds of industrial interest, such as formic acid and acetic acid. For this, the authors started with solutions of acetol, FeCl₃ and H₂O₂, all with defined concentrations, using acetonitrile as solvent in a batch reactor, obtaining satisfactory amounts of acetic acid, formic acid and CO₂. In the present invention, in the homogeneous process, the production of compounds of industrial interest does not require the addition of a catalyst, only hydrogen peroxide, since the substrate impurities act as a catalyst. Furthermore, unlike the cited paper, the solvent used is water.

The paper “Nb and V-modified silicate for conversion of glycerol: Comparison between the waste and commercial product”, Catalysis Today, volume 289, 22 Sep. 2016, p. 258-263, depicts obtaining products with high added value, using both commercial glycerin and residual glycerin from biodiesel production as substrate, via heterogeneous catalysis. Unlike this invention, the reactions were carried out in a batch reactor, using Nb and V catalysts supported on silica, obtaining as main products allyl alcohol, when commercial glycerin was used as substrate, and acetone, when residual glycerin was used as substrate.

The paper “Oxidation of bio-renewable glycerol to value-added chemicals through catalytic and electro-chemical processes”. Applied Energy, volume 230, 2 Sep. 2018, 1347-1349, addresses to a review which presents some works that studied the conversion of the glycerol molecule into different products. Due to the versatility of this molecule, several products can be obtained, varying the process (homogeneous, heterogeneous, electrochemical catalysis, etc.) and the type of catalyst used.

In “Synthetic niobium oxyhydroxide as a bifunctional catalyst for production of ethers and allyl alcohol from waste glycerol”, Journal of the Brazilian Chemical Society, volume 28, 2017, p. 2244-2253, the synthesis of a new niobium catalyst and its use in reactions converting residual glycerol into value-added products is reported. In the reactions, a batch reactor, niobium catalysts, hydrogen peroxide as oxidizing agent, and residual glycerol were used, preferably obtaining allyl alcohol and cyclic ethers. The difference between this work and the invention in question refers to the way in which the reactions via heterogeneous catalysis were carried out. In the case of this invention, mesoporous silica was used as catalyst, without the need to incorporate a metal to increase its activity, and the reactions were carried out in a reactor that operates in continuous flow, simulating an industrial process.

The paper “Zirconium-modified mesoporous silica as an efficient catalyst for the production of fuel additives from glycerol”. Catalysis Communications, volume 110, 26 Feb. 2018, p. 1-4, addresses to the acetylation reaction of the glycerol molecule using acetic acid. For the reactions, a mesoporous silica catalyst modified with zirconium was used, and the glycerol conversion and selectivity for diacylglycerol and triacylglycerol were evaluated as a function of time.

The cited works make use of supported catalysts and, in no case, modify the form of synthesis/preparation of the support, mainly to favor the formation of compounds such as metalate peroxides. They also do not address to obtaining products through catalysts without immobilization of other elements as an active phase, for example.

In general, the works present in the State of the Art employ only commercial glycerin, not mentioning ways and technologies capable of using residual glycerin, from the production of biodiesel.

Patent PI0803767-1 (Production process of glycerin mono ethers and its application as an additive for biodiesel) addresses to processes that report catalytic reactions of glycerin conversion. The work describes the selective production of glycerin mono ethers using solid acidic catalysts, such as zeolite. The employed method uses organic solvents and drastic reaction conditions, which would make the industrial process quite costly.

Patents 9304714-Degussa (Process for the preparation of acrolein) and US 20080183019 (Novel catalysts and process for dehydrating glycerol) describe the use of tungsten-based compounds, and the process is carried out at temperatures between 180 to 350° C. and pressures between 0.1 and 200 atm (101.325 kPa and 20.265 MPa).

Other patents further report production processes for 1,3-propanediol; among them, PI9910519 (Process for bioproduction of 1,3-propanediol and transformed host cell), U.S. Pat. No. 5,821,092 (Production of 1,3-propanediol from glycerol by recombinant bacteria expressing recombinant diol dehydratase), U.S. Pat. No. 5,633,362 (Production of 1,3-propanediol from glycerol by recombinant bacteria expressing recombinant diol dehydratase). Such processes use microorganisms to convert glycerol to 1,3-propanediol.

The process developed by the company Shell, holder of the US patent U.S. Pat. No. 6,080,898 (Hydrogenolysis of glycerol), uses homogeneous catalysts based on platinum or a metallic compound from the platinum group. EP 1853541B1 (Process for dehydrating glycerol to acrolein) reports the process of producing acrolein by dehydrating glycerol in the presence of molecular oxygen.

The most used route in the conversion of glycerin involves homogeneous catalysis, using thermal treatment, in the presence of inorganic acids or bases, which makes the process less selective, forming different products with low selectivity.

The method applied in this invention uses residual glycerin from biodiesel production. Obtaining some products of this invention uses silica catalyst (without immobilization of other elements as active phase) and unprecedented experimental conditions. In addition, the reaction system employed is not reported in the State of the Art, and employs residual glycerin conversion reactions in two different systems, semi-batch and continuous flow, using homogeneous and heterogeneous catalysis.

The reaction system and the catalyst, as well as the reaction products obtained in this invention, are different from those presented in the State of the Art.

The method described in the present invention is innovative in the following points: (i) use of mild temperature/pressure conditions and use of pure silica heterogeneous catalyst, without the need to incorporate an active phase, i.e., of metals; (ii) use of the impurities present in the residual glycerin as a catalyst in the conversion of glycerol via homogeneous catalysis, adding only one oxidizing agent; (iii) obtaining, with high selectivity, compounds with petrochemical applications, mainly formic acid and ethers; (iv) reaction carried out without the use of organic solvents, which makes the process less costly and environmentally friendly.

### BRIEF DESCRIPTION OF THE INVENTION

The present invention consists of steps, which involve the development of the pure silica catalyst and conversion reactions of residual and/or commercial glycerin using an oxidizing agent, via homogeneous and heterogeneous catalysis.

### BRIEF DESCRIPTION OF THE DRAWINGS

The present invention will be described in greater detail below, with reference to the attached figures which, in a schematic way and not limiting the inventive scope, represent examples of its embodiment. The figures are:

FIG. 1, which represents: Infrared absorption spectrum for the SiO₂ catalyst, a) after pyridine adsorption, and b) corresponds to the result after addition of H₂O₂ and subsequent pyridine adsorption. Raman spectrum for SiO₂ catalyst, c) before the treatment with H₂O₂ and d) corresponds to the result after the treatment with H₂O₂. In the Raman spectra, bands 1, 2 and 3 were attributed to the different Si—O—Si vibrations and band 4 was attributed to a new bond between the silicon atom and the oxygen deposited by H₂O₂, in vacant sites to SiO₂.

FIG. 2 represents a general schematic of the continuous flow reactor. The system consists of a loading vessel (1), where the reaction mixture, residual glycerin and peroxide, is added, which is taken to the reactor through a pump (2). Before the reactor inlet, there is a safety valve (3), which helps to control the internal pressure of the reactor. The feed is provided by the lower part of the reactor (4) and in the upper part (5) there are 3 (three) thermocouples (6a, 6b, 6c), which make it possible to control the temperature in the different zones of the reactor. The reactor, which is inside an oven during the entire reaction, is divided into three parts, zone I, filled with an inert material (silicon carbide), is the place for preheating the reagents, zone II, in which a mixture of silicon carbide and catalyst is placed and where the reaction takes place, and zone III, which is also filled with silicon carbide only. On the side of the reactor, at the end of zone III, there is a gas outlet (7), coupled to a condenser (8), which liquefies the reaction products and these are collected in a collection flask (9) throughout the reaction.

FIG. 3, which represents the glycerol conversion and selectivity data for one of the reaction products using the SiO₂ catalyst. The bars represent the percentages of glycerol conversion and the solid line with squares, the percentages of selectivity for formic acid.

FIG. 4, which represents the glycerol conversion and selectivity data for the main reaction products. The bars represent the percentages of glycerol conversion, the solid line with squares, the percentages of selectivity for formic acid, the solid line with circles, the percentages of selectivity for diglycerol, and the solid line with triangles, the percentages of selectivity for dioxanes.

FIG. 5, which illustrates the schematic representation of a reactor in an experimental reflow system, used in residual glycerin conversion reactions. There are identified: outlet of products (a), addition of H₂O₂ (b), condenser (8), magnetic plate (10), volumetric flask (11).

FIG. 6, which illustrates the conversion data of residual (e) and commercial (f) glycerin, and selectivity for the main reaction products in a reflow system. The bars represent the percentages of glycerol conversion. In (e) and (f), the solid line with squares represents the percentages of selectivity for formic acid, the solid line with circles, the percentages of selectivity for diglycerol, the solid line with triangles, the percentages of selectivity for dioxanes, and the solid line with diamonds, the percentages of selectivity for hydroxypropanone.

### DETAILED DESCRIPTION OF THE INVENTION

The present invention addresses to a method of obtaining compounds of high added value, from residual glycerin, derived from the synthesis of biodiesel, or commercial. The glycerin conversions carried out in this invention occur through two catalytic processes: (i) heterogeneous catalysis, using a synthetic silica catalyst (without the use of metals), with acidic properties and presenting mesopores; (ii) homogeneous catalysis, where the catalyst used constitutes an impurity of the residual glycerin, adding only an oxidizing agent, such as benzoyl peroxide or hydrogen peroxide (H₂O₂).

The catalytic conversion reactions, object of this invention, were carried out in a continuous flow reactor (fixed-bed reactor, PBR) and in a semi-batch system, at a temperature of 100 to 250° C.

In flow and semi-batch systems, residual and commercial glycerin were tested. In homogeneous catalysis, the main reaction products in the semi-batch reactor, using residual glycerin, were formic acid and 1-hydroxypropanone. On the other hand, in the reaction using commercial glycerin, diglycerol was obtained as the major product. In PBR, both residual and commercial glycerines were converted into dioxanes (cyclic ethers).

Still in the case of homogeneous catalysis, it is worthy to highlight that, even in the absence of the catalyst, only in the presence of the oxidizing agent, it is possible, with the method employed in this invention, to promote the conversion of residual and commercial glycerin by the action of remaining components of biodiesel synthesis.

The reaction is also carried out by adding sodium methylate to convert commercial glycerin. Sodium methylate provides the methoxide anion (proving complementary homogeneous catalytic action) when using commercial glycerin.

In heterogeneous catalysis, the use of synthetic silica-based catalysts promotes the effective conversion of residual glycerin from biodiesel production into formic acid with high selectivity and stability, in a continuous flow reaction.

The method described in this invention allows obtaining compounds with high selectivity and the reactions, via heterogeneous or homogeneous catalysis, are performed without the need to use organic solvents, which makes the process less costly and environmentally friendly.

The catalytic conversion method of glycerin developed in this invention involves the production steps of pure silica catalyst, in the case of heterogeneous catalysis, followed by conversion reactions. In the homogeneous catalysis of residual and/or commercial glycerin, the products formed are formic acid and green ethers (such as diglycerol, cyclic ethers and/or 1-hydroxypropanone), so named because they are derived from residual glycerol from biodiesel synthesis, using 35 to 50% peroxides as oxidizing agents. In heterogeneous catalysis, there is the use of heterogeneous catalyst formed by pure synthetic silica (SiO₂) with high specific surface area (>1000 m² g⁻¹) and presence of acidic groups.

Both reactions take place at a temperature of 100 to 250° C. in a continuous flow reactor and/or in a semi-batch system.

The present invention can be better understood through the steps highlighted below, which involve the development of the pure silica catalyst and the glycerin conversion reactions via homogeneous and heterogeneous catalysis.

Step 1: Synthesis of pure silica catalyst

A solution of NaOH and CTAB (Cetyltrimethylammonium bromide) is added to a beaker. In the resulting mixture, a solution of TEOS (Tetraethylortosilicate) is slowly dripped, and the system is left under magnetic stirring. The formation of a white solid is observed, which is filtered under vacuum and washed with distilled water until neutral pH. After filtration and washing, the solid is taken to the oven, subsequently macerated and subjected to a thermal treatment, following a heating ramp.

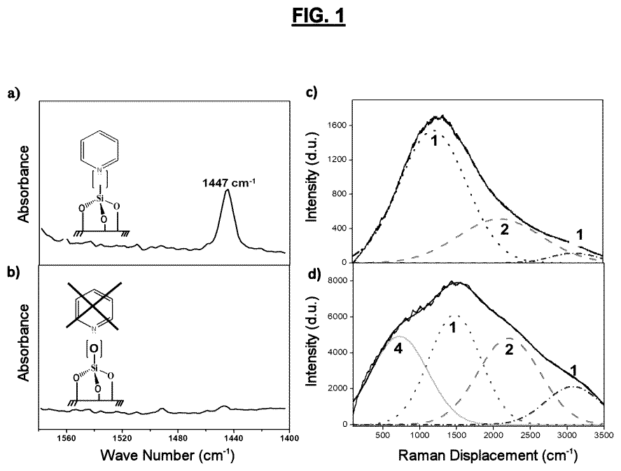

The catalyst obtained in Step 1 was subjected to several analyses in order to characterize the material. To determine the acidity, the surface of the compound was subjected to cleaning, at a temperature of 150° C., under N₂ flow and subsequent adsorption of pyridine. The interaction between this basic molecule and the acidic sites of the catalyst is evaluated by the presence of a specific absorption band in the Infrared region, as shown in FIG. 1a, wherein the 1447 cm⁻¹ band is related to the interaction between the pyridine molecule and the Lewis acidic sites. These Lewis acidic sites are associated with the presence of vacant oxygen sites on some silicon atoms.

The oxidizing character of the catalyst is related to its ability to decompose H₂O₂ and the consequent deposition of an oxygen atom in vacant sites of silicon atoms, or vacant sites of oxygen, as represented in FIG. 1b. The presence of this oxygen atom on the Lewis vacant site was proposed from the analyses of the absorption spectrum in the Infrared region for the sample, after the addition of H₂O₂ and subsequent adsorption of pyridine (FIG. 1b), and the Raman spectrum of the catalyst before the addition (FIG. 1c), and after the addition of H₂O₂ (FIG. 1d). It was observed that the sample, after being in contact with H₂O₂, did not present Lewis acidic sites, indicating a possible occupation of these vacant sites. In addition, after the addition of H₂O₂ to the catalyst, the presence of a band in the Raman was observed, which was attributed to a new bond between the silicon atom and the oxygen (active species) deposited after the decomposition of H₂O₂. The other bands (1, 2 and 3) in the Raman spectra are the same for the material before and after the addition of H₂O₂ and were attributed to the different Si—O—Si vibrations (FIGS. 1c and 1d).

### Step 2: Catalytic Tests for Conversion of Residual Glycerin Using Pure Silica Catalyst: Reactions in Continuous Flow.

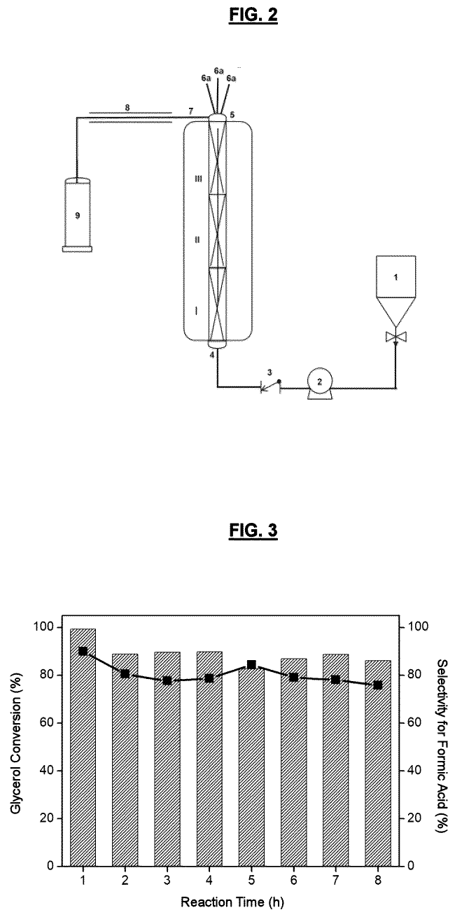

The reactions in continuous flow carried out in this invention occur in a reactor that remains inside an oven throughout the process, as shown in FIG. 2. In the load vessel (1), there is the reaction mixture that is taken to the reactor through a pump (2). The feed is provided by the lower part of the reactor (4) and in the upper part (5) there are 3 (three) thermocouples (6a, 6b, 6c) that allow the control of the temperature in the different zones of the reactor. The reactor is divided into three parts, zone I, which is filled with an inert material, silicon carbide; in zone II, a mixture of silicon carbide and catalyst is placed, and zone III is also filled only with silicon carbide. On the side, at the end of zone III, there is a gas outlet (7), coupled to a condenser (8), which liquefies the reaction products, which are collected in a collection flask (9) during the entire reaction.

For the reaction in question, zones I and III of the reactor are filled with silicon carbide and zone II is filled with a solid mixture of silicon carbide and catalyst. The reaction mixture consists of a residual glycerine and hydrogen peroxide solution, with a feed flow of 1 mL·min⁻¹ to the reactor.

At intervals of one hour, the volume and mass of the product formed during this period were determined, with the maximum reaction time in the test corresponding to 8 hours. Collected samples taken during the reaction were analyzed by GC-MS. Conversion and selectivity were determined from a calibration curve.

The pure silica catalyst, called SiO₂, showed a constant conversion (approximately 90%) during the 8 uninterrupted hours of reaction, suggesting that the catalyst has a high stability in the reaction of conversion of residual glycerin. In addition, the SiO₂ catalyst was able to promote the conversion of glycerol with high selectivity to formic acid (approximately 80%) during the entire time studied, as can be seen in FIG. 3.

Since formic acid is an oxidative cleavage product of glycerol, the catalyst in question is capable of promoting dehydration and oxidation of the glycerol molecule.

### Step 3: Catalytic Tests for Conversion of Residual Glycerin Via Homogeneous Catalysis

The transesterification of triglycerides for the production of biodiesel generates as a co-product a glycerin that undergoes a neutralization process, giving rise to the blond or residual glycerin exported by the biodiesel production units, which is the main component of the reactions of the present invention. The impurities present in residual glycerin, in the presence of an oxidizing agent such as H₂O₂, are capable of converting the same into products of commercial interest, via homogeneous catalysis. This result was observed using only residual glycerin and H₂O₂, in a continuous flow system. The reaction was carried out in a continuous flow reactor described in Step 2.

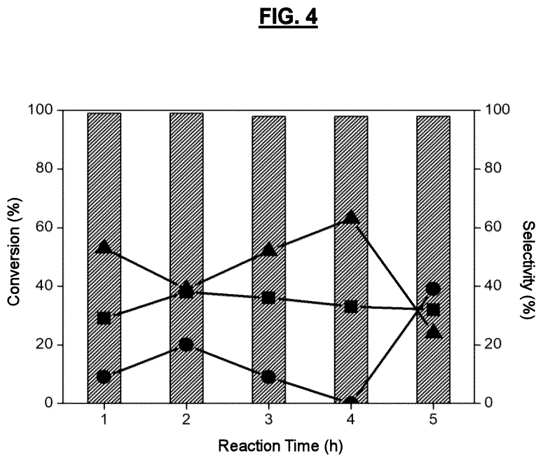

Products such as formic acid, diglycerol and dioxanes were identified in the GC-MS as products of this reaction and their respective selectivities are shown in FIG. 4.

Dioxanes are the major products of this reaction and are compounds that can be used as additives in biodiesel formulation, improving its properties at low temperatures and reducing its viscosity. Then, the formation of formic acid is observed, which is a product of the oxidation of glycerol and stands out for its wide application in the textile, agricultural, pharmaceutical and chemical industries. Currently, formic acid is used as a hydrogen storage compound, since it can be decomposed into hydrogen and CO₂. In lower concentration, diglycerol can be observed as a product of the reaction. Diglycerol is formed due to the etherification of glycerol and has numerous applications in the food, pharmaceutical and cosmetics industries.

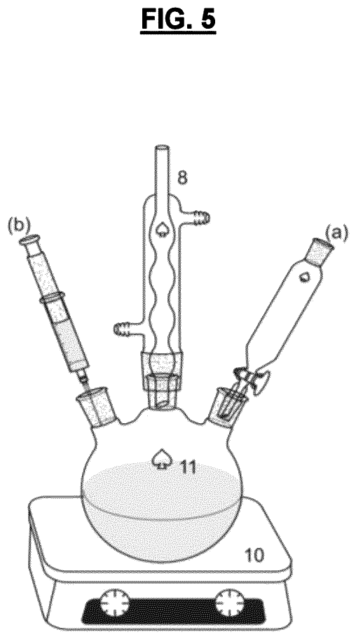

Reflow reactions were employed to study reactions in a semi-batch system. Furthermore, these reactions allowed us to understand the formation mechanism of its main products. The reactions at reflow were carried out according to the system shown in FIG. 5.

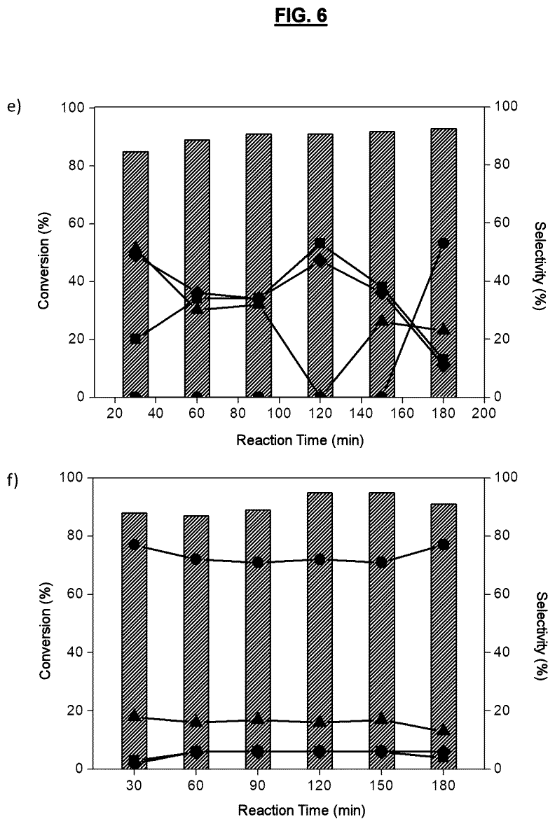

The reaction mixture consisted of residual or commercial glycerin and H₂O₂ solution. The reactions took place at 150° C. for 3 hours and, every 30 minutes, aliquots of the sample were taken and taken for analysis in the GC-MS. The results are shown in FIG. 6.

In a system at reflow, the conversion of residual glycerin presents as main products formic acid, hydroxypropanone and dioxanes, as observed in FIG. 6a. Diglycerol is only formed at the end of the reaction, after 160 minutes. The relative selectivity for the production of formic acid gradually increases up to 120 minutes, reaching 55% selectivity. The same behavior can be observed for hydroxypropanone. After this time, the selectivity reduces and there is an increase in selectivity for diglycerol, reaching 60%. The presence of formic acid can be explained by the oxidative cleavage mechanism of glycerol with the aid of the oxidizing agent (H₂O₂). The formation of formic acid from glycerol can be explained firstly by the dehydration of glycerol, forming hydroxypropanone, and then by the generation of formic acid from the oxidation of hydroxypropanone and/or direct oxidation of glycerol. Hydroxypropanone can be obtained from glycerol via dehydration. This substance is an important intermediate used in the production of polyols and acrolein. In addition, it can be used in the textile industry, in cosmetics and as flavoring agents.

In FIG. 6b, the formation of diglycerol is observed as the major product, around 70%, in the reflow reaction of conversion of commercial glycerol. Dioxane, formic acid and hydroxypropanone molecules were also identified as minor products in the reaction (˜10%). The main product is probably obtained by the oligomerization of glycerol in the presence of an oxidizing agent, such as hydrogen peroxide, in the reaction medium.

Comparing the results presented, it is clear that there is a preference in the formation of formic acid and diglycerol using residual and commercial glycerin, respectively. This difference in the products obtained is probably due to impurities present in glycerin (such as sodium chloride and methanol) which, when reacting with hydrogen peroxide, generate species responsible for dehydrating and oxidizing the glycerol molecule, forming hydroxypropanone and formic acid.

As the reaction progresses, sodium chloride is consumed, there is the reduction in the formation of these products, and the increase in diglycerol is observed. The production of diglycerol, in turn, is directly related to the reaction of hydrogen peroxide with glycerol, resulting in the oligomerization of glycerin. This effect occurs both for residual glycerin and for commercial glycerin; however, it is more significant in commercial glycerin, due to the fact that it does not have impurities. In both situations, starting from glycerin as a by-product of biodiesel production, it is highlighted the obtention of industrially important products, in the absence of a heterogeneous catalyst and in mild reaction conditions.

This invention shows the importance of using hydrogen peroxide as a green oxidizing agent in glycerol conversion reactions, but is not restricted to the same. Hydrogen peroxide has low cost and high availability, which makes it very attractive, making the glycerin conversion process a simple and inexpensive process.

1. A method of catalytic conversion of glycerin into high added value products, the method comprising:
a) Homogenizing the residual glycerin containing salts and impurities in peroxide solution by means of a static mixer;
b) Pumping the solution obtained in a) to a reactor containing silica catalyst inside;
c) Leaving the mixture b) in the reactor for a residence time of 2 hours; and
d) Collecting the products obtained in c) and separating them by distillation.

2. The method according to claim 1, wherein it comprises the synthesis steps of a pure silica catalyst with a high specific surface area (>1000 m2 g−1) and presence of acidic groups, applied in the process of heterogeneous catalysis.

3. The method according to claim 1, wherein it employs 35 to 50% of peroxides as oxidizing agents, such as, but not restricted to, hydrogen peroxide (H₂O₂) and benzoyl peroxide, in the process of homogeneous catalysis.

4. The method according to claim 1, wherein characterized in that the residual glycerin comes from the process of obtaining biodiesel.

5. The method according to claim 1, wherein it promotes the conversion of residual glycerin into formic acid, via heterogeneous catalysis, at a temperature of 100 to 250° C.

6. The method according to claim 5, wherein it promotes the conversion of residual glycerin into formic acid, via heterogeneous catalysis, using a continuous flow reactor, also being used batch or semi-batch reactors.

7. The method according to claim 1, wherein it promotes the conversion of residual glycerin into formic acid and green ethers, via homogeneous catalysis, at a temperature of 100 at 250° C.

8. The method according to claim 7, wherein it promotes the conversion of residual glycerin into formic acid and green ethers, via homogeneous catalysis, using a continuous flow reactor or in a semi-batch system.

9. The method according to claim 1, wherein the conversion of residual glycerin by homogeneous catalysis occurs by the action of components remaining from the synthesis of biodiesel, with the formation of major compounds such as formic acid, cyclic ethers and diglycerol.

10. The method according to claim 1, wherein it promotes the conversion of commercial glycerin into formic acid and green ethers, via homogeneous catalysis, at a temperature of 100 to 250° C.

11. The method according to claim 10, wherein it promotes the conversion of commercial glycerin into formic acid and green ethers, via homogeneous catalysis, using a continuous flow reactor or in a semi-batch system.

12. The method according to claim 1, wherein it adds sodium salts before step a) of homogenization of commercial glycerin with peroxide solution.

13. The method according to claim 12, wherein it employs inorganic or organic salts.

14. (canceled)

15. The method according to claim 13, wherein the inorganic or organic salts are selected from the group consisting of sodium chloride and sodium methylate.

16. The method according to claim 13, wherein the inorganic or organic salts are dissolved in the glycerin, in water, or in methanol.
